(19) United States
(12) Patent Application Publication
(10) Pub. No.: US 2025/0000947 A1
(43) Pub. Date: Jan. 2, 2025

(54) OXYTOCIN CONJUGATES FOR TREATING NEONATAL DISORDERS

(71) Applicant: Elgan Pharma Ltd, Nazareth (IL)

(72) Inventors: Michal OLSHANSKY, Tel Aviv (IL); Elena OSTROVSKY, Rishon-LeZion (IL); Stav ZELDIS, Herzeliya (IL)

(73) Assignee: Elgan Pharma Ltd, Nazareth (IL)

(21) Appl. No.: 18/693,220

(22) PCT Filed: Sep. 19, 2022
(86) PCT No.: PCT/IL2022/051002

§ 371 Date: Mar. 19, 2024

Related U.S. Application Data
(60) Provisional application No. 63/245,890, filed on Sep. 19, 2021.

(51) Int. Cl. A61K 38/22; A61K 9/00; A61K 47/54; A61K 47/69

(52) U.S. Cl. CPC A61K 38/22; A61K 9/0043; A61K 47/542; A61K 47/6951

(57) ABSTRACT

A composition-of-matter including oxytocin, an oxytocin analog or derivative or an oxytocin receptor agonist bound to a fatty acid and methods of using the composition in treating neonate disorders. Neonate disorders include Neonatal Abstinence Syndrome (NAS), Prader Willie Syndrome, sleep disorders, seizures, and feeding disorders by delivering a conjugate including oxytocin, an oxytocin analog or derivative or an oxytocin receptor agonist bound to a fatty acid to the neonate.

### RELATED APPLICATION/S

This application claims the benefit of priority of U.S. Provisional Patent Application No. 63/245,890 filed on 19 Sep. 2021, the contents of which are incorporated herein by reference in their entirety.

### BACKGROUND

The present invention relates to pharmaceutical compositions for treating neonatal disorders such as neonatal abstinence syndrome (NAS). Embodiments of the present invention relate to an oxytocin-fatty acid conjugate formulated for nasal delivery in neonates.

Neonatal abstinence syndrome (NAS) is a group of conditions that result from sudden discontinuation of fetal exposure to substances such as opioids, antidepressants, barbiturates or benzodiazepines that were taken by the mother during pregnancy.

The pathophysiology of NAS is not completely understood. The Finnegan scoring system is commonly used to assess the severity of NAS and is used for initiating, monitoring, and terminating treatment in neonates. Morphine is the most commonly used drug in the treatment of NAS secondary to opioids.

Oxytocin is a cyclic nonapeptide hormone with the amino acid sequence CYIQNCPLG. Oxytocin acts as a neurotransmitter in the brain and is the principal uterine-contracting and milk-ejecting hormone of the posterior pituitary.

Use of Oxytocin in treating NAS has been previously described (US20200316162). It has been suggested that intranasally administered oxytocin reaches the central nervous system and increases central concentrations of oxytocin allowing increased activity at central oxytocin receptors. This transport is purported to occur via ensheathed channels surrounding olfactory and trigeminal nerve fibers after nasal spray deposition onto the olfactory and respiratory epithelia.

While animal studies have shown that oxytocin can be used to reduce or ameliorate symptoms associated with NAS (US20200316162), there remains a need for oxytocin compositions that exhibit increased brain bioavailability upon intranasal delivery (or via a different delivery route) and thus can be safely used to treat neonatal disorders such as NAS.

### SUMMARY

According to one aspect of the present invention there is provided a composition-of-matter comprising a conjugate of oxytocin, an oxytocin analog or derivative or an oxytocin receptor agonist bound to a fatty acid.

According to embodiments of the present invention the fatty acid is Docosahexaenoic acid (DHA).

According to embodiments of the present invention the DHA is bound to the oxytocin, oxytocin analog or derivative or an oxytocin receptor agonist through an amide linkage.

According to embodiments of the present invention a weight ratio of oxytocin, an oxytocin analog or derivative or an oxytocin receptor agonist to fatty acid is 1:3000 to 1:200.

According to embodiments of the present invention the composition-of-matter further comprises a cyclodextrin.

According to embodiments of the present invention the conjugate is trapped in or bound to, an inclusion complex formed from the cyclodextrin.

According to embodiments of the present invention the cyclodextrin is (2-Hydroxypropyl)-β-cyclodextrin.

According to embodiments of the present invention a weight ratio of the conjugate to the cyclodextrin is 1:60,000 to 1:600.

According to embodiments of the present invention the composition-of-matter further comprises liposomes encapsulating the conjugate.

According to another aspect of the present invention there is provided a pharmaceutical composition comprising a conjugate including oxytocin, an oxytocin analog or derivative or an oxytocin receptor agonist bound to a fatty acid and a carrier for mucosal delivery.

According to embodiments of the present invention the carrier is formulated for nasal delivery.

According to embodiments of the present invention the pharmaceutical composition further comprises a cyclodextrin forming an inclusion complex.

According to embodiments of the present invention the conjugate is trapped in or bound to the inclusion complex.

According to embodiments of the present invention the pharmaceutical composition further comprises liposomes encapsulating the conjugate.

According to another aspect of the present invention there is provided a nebulizer for nasal delivery of the pharmaceutical composition.

According to another aspect of the present invention there is provided method of treating Neonatal Abstinence Syndrome (NAS) comprising delivering a conjugate including oxytocin, an oxytocin analog or derivative or an oxytocin receptor agonist bound to a fatty acid to a neonate having NAS.

According to another aspect of the present invention there is provided a method of reducing the frequency and severity of Neonatal Abstinence Syndrome (NAS) symptoms including trembling, excessive crying or high-pitched crying, sleep problems, tight muscle tone, overactive reflexes, seizures, yawning, stuffy nose, and sneezing, poor feeding and sucking, vomiting or diarrhea, sweating, fever or unstable temperature, by delivering a conjugate including oxytocin, an oxytocin analog or derivative or an oxytocin receptor agonist bound to a fatty acid to a neonate having NAS.

According to another aspect of the present invention there is provided a method of reducing overall Finnegan score by delivering a conjugate including oxytocin, an oxytocin analog or derivative or an oxytocin receptor agonist bound to a fatty acid to a neonate having NAS.

According to another aspect of the present invention there is provided a method of reducing the need and dosage of narcotics prescribed to an infant with NAS (including methadone, morphine and buprenorphine), by delivering a conjugate including oxytocin, an oxytocin analog or derivative or an oxytocin receptor agonist bound to a fatty acid to a neonate having NAS.

According to another aspect of the present invention there is provided a method of reducing the duration of hospital stay by delivering a conjugate including oxytocin, an oxytocin analog or derivative or an oxytocin receptor agonist bound to a fatty acid to a neonate having NAS.

According to another aspect of the present invention there is provided a method of treating other pediatric disorders including Prader Willie Syndrome, sleep disorders, seizures, and feeding disorders, by delivering a conjugate including oxytocin, an oxytocin analog or derivative or an oxytocin receptor agonist bound to a fatty acid to a neonate having NAS.

According to embodiments of the present invention the conjugate is trapped in or bound to an inclusion complex formed by a cyclodextrin.

According to embodiments of the present invention the conjugate is encapsulated by a liposome.

According to embodiments of the present invention delivery is effected using a nebulizer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates one embodiment of the oxytocin-DHA conjugate of the present invention.

FIG. 2 is an HPLC chromatogram of Oxytocin, free form.

FIG. 3 is a magnification of FIG. 2 showing the oxytocin degradation products.

FIG. 4 is an HPLC chromatogram of the reaction mixture of the present invention.

FIGS. 5 and 6 are HPLC chromatograms of formulations 1 and 2 (respectively).

### DETAILED DESCRIPTION

The present invention is of an oxytocin conjugate which can be used to treat neonatal disorders. Specifically, the present invention can be used to intranasally treat neonates having Neonatal abstinence syndrome (NAS).

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Neonatal abstinence syndrome (NAS) is a group of conditions that result from sudden discontinuation of fetal exposure to substances such as opioids, antidepressants, barbiturates or benzodiazepines that were taken by the mother during pregnancy.

Symptoms of NAS include: trembling, excessive crying or high-pitched crying, sleep problems, tight muscle tone, overactive reflexes, seizures, yawning, stuffy nose, and sneezing, poor feeding and sucking, vomiting or diarrhea, sweating, fever or unstable temperature. The Finnegan Neonatal Abstinence Scoring System is the most commonly used scoring tool, although the original tool has been modified frequently. Specific drugs have been linked to specific complications in the baby, including Heroin and other opioids, including methadone, and Amphetamines and cocaine. Opioids can cause serious withdrawal symptoms in the newborn. Some symptoms can last as long as 4 to 6 months. Seizures may also occur in babies born to opioid users. Amphetamines can lead to low birth weight and premature birth. Cocaine use can cause poor growth and can also lead to complications such as placental abruption more likely.

At present, morphine and methadone are the most commonly used drug in the treatment of NAS secondary to opioids. Despite recommendations that opioids should be used for treatment of NAS and NAS symptoms, no universal evidence-based pharmacologic treatment strategy exists. In addition, no drug is approved for use in infants by the FDA (Davis, 2018). Most drugs used to treat newborns are adult formulations that contain preservatives and alcohol that have not been proven to be safe and could affect neurodevelopmental outcome (Davis, 2018). The use of these drugs in neonates is controversial due to adverse events such as shallow breathing, apnea, hypotension, bradycardia, oxygen desaturation, lethargy, poor feeding, hypothermia, and emesis (Davis, 2018).

Oxytocin has been suggested as an alternative to morphine in treating NAS. While oxytocin treatment lacks the side effects typically associated with morphine, the challenge is a neonatal-safe formulation that is suitable for neonatal administration.

Thus, according to one aspect of the present invention there is provided a composition-of-matter suitable for treating neonatal disorders such as Prader Willie Syndrome or NAS. Additional disorders include sleep disorders, seizures, and feeding disorders, associated with NAS, PWS, or other complications leading to prolonged inability to thrive.

The present composition includes oxytocin, an oxytocin analog or derivative or an oxytocin receptor agonist bound to a fatty acid to form a conjugate.

Oxytocin is a peptide hormone having the amino acid sequence CYIQNCPLG.

The term “peptide” as used herein encompasses native peptides, synthetic peptides or recombinant peptides, peptidomimetics and the peptide analogues peptoids and semipeptoids, and may have, for example, modifications rendering the peptides more stable while in a body or more capable of penetrating into cells. Such modifications include, but are not limited to: N-terminus modifications; C-terminus modifications; peptide bond modifications, including but not limited to CH₂—NH, CH₂—S, CH₂—S—O, O—C—NH, CH₂—O, CH₂—CH₂, S═C—NH, CH═CH, and CF═CH;

backbone modifications; and residue modifications. Methods for preparing peptidomimetic compounds are well known in the art and are specified, for example, in Ramsden, C. A., ed. (1992), Quantitative Drug Design, Chapter 17.2, F. Choplin Pergamon Press, which is incorporated by reference as if fully set forth herein. Further details in this respect are provided hereinbelow.

Peptide bonds (—CO—NH—) within the peptide may be substituted, for example, by N-methylated bonds (—N(CH₃)—CO—); ester bonds (—C(R)H—C—O—O—C(R)—N—); ketomethylene bonds (—CO—CH₂—); α—aza bonds (—NH—N(R)—CO—), wherein R is any alkyl group, e.g., methyl; carba bonds (—CH₂—NH—); hydroxyethylene bonds (—CH (OH)—CH₂—); thioamide bonds (—CS—NH—); olefinic double bonds (—CH═CH—); retro amide bonds (—NH—CO—); and peptide derivatives (—N(R)—CH₂—CO—), wherein R is the “normal” side chain, naturally presented on the carbon atom. These modifications can occur at any of the bonds along the peptide chain and even at several (2-3) at the same time.

Natural aromatic amino acids, Trp, Tyr, and Phe, may be substituted for synthetic non-natural acids such as, for instance, tetrahydroisoquinoline-3-carboxylic acid (TIC), naphthylelanine (Nol), ring-methylated derivatives of Phe, halogenated derivatives of Phe, and o-methyl-Tyr.

In addition to the above, the peptides of the present invention may also include one or more modified amino acids or one or more non-amino acid monomers (e.g., fatty acids, complex carbohydrates, etc.).

The term “amino acid” or “amino acids” is understood to include the 20 naturally occurring amino acids; those amino acids often modified post-translationally in vivo, including, for example, hydroxyproline, phosphoserine, and phosphothreonine; and other less common amino acids, including but not limited to 2-aminoadipic acid, hydroxylysine, isodesmosine, nor-valine, nor-leucine, and ornithine. Furthermore, the term “amino acid” includes both D- and L-amino acids.

Oxytocin is preferably utilized herein in the native oxidized octapeptide oxytocin disulfide form (cyclic). However, the reduced straight-chain (non-cyclic) dithiol nonapeptide oxytoceine may also be used since it may be re-oxidized to oxytocin via the dehydroascorbate/ascorbate redox couple.

The peptides of the present invention may be synthesized by any techniques that are known to those skilled in the art of peptide synthesis. For solid phase peptide synthesis, a summary of the many techniques may be found in: Stewart, J. M. and Young, J. D. (1963), “Solid Phase Peptide Synthesis,” W. H. Freeman Co. (San Francisco); and Meienhofer, J (1973). “Hormonal Proteins and Peptides,” vol. 2, p. 46, Academic Press (New York). For a review of classical solution synthesis, see Schroder, G. and Lupke, K. (1965). The Peptides, vol. 1, Academic Press (New York).

Further description of peptide synthesis is disclosed in U.S. Pat. No. 6,472,505. A preferred method of preparing the peptide compounds of the present invention involves solid-phase peptide synthesis, utilizing a solid support. Large-scale peptide synthesis is described by Andersson Biopolymers 2000, 55 (3), 227-50.

As is mentioned hereinabove, oxytocin analogs, derivatives or receptor agonists having oxytocin activities in the brain can also be used in the present conjugate.

The fatty acid used in the present conjugate can be any saturated or unsaturated fatty acid. Examples include, but are not limited to, arachidic acid (ARA), linoleic acid (LA), Gamma linolenic acid (GLA), Conjugated linoleic acid (CLA), myristic acid, oleic acid, capric acid, alpha-linolenic acid (ALA), eicosapentaenoic acid (EPA), or docosahexaenoic acid (DHA).

The oxytocin, oxytocin analog, derivative or receptor agonist can be bound to the fatty acid using one of several approaches. Examples 1 and 2 describe approaches for conjugating oxytocin to DHA.

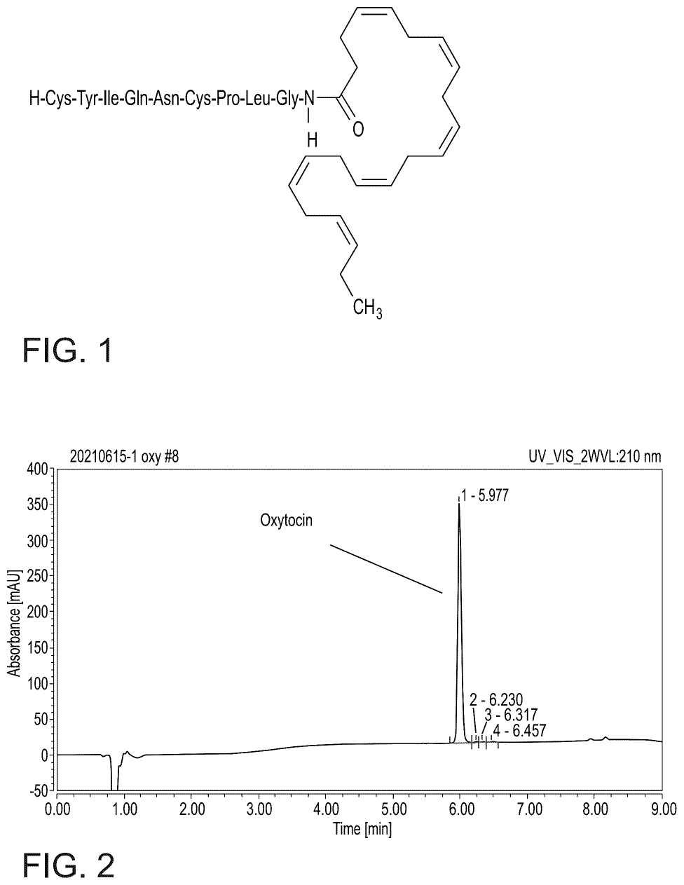

FIG. 1 illustrates the chemical structure of one embodiment of the present conjugate (i.e. human oxytocin conjugate with cis-4,7,10,13,16,19-Docosahexaenoic acid). Empirical formula of human oxytocin is C₄₃H₆₆N₁₂O₁₂S2, MW 1007.19 g/mol.

Empirical formula of cis-4,7,10,13,16,19-Docosahexaenoic acid is C₂₂H₃₂O₂, MW 328.49 g/mol. Empirical formula of human oxytocin conjugate with cis-4,7,10,13,16,19-Docosahexaenoic acid is C₆₅H₉₇N₁₂O₁₃S2, MW 1317.68 g/mol.

As is mentioned hereinabove, the present conjugate can be used to treat neonatal disorders such as NAS.

The conjugate of the present invention can be administered to a neonate per se, or in a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the conjugate (e.g., oxytocin-DHA) accountable for the intended biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound.

Herein, the term “excipient” refers to an inert substance added to a pharmaceutical composition to further facilitate administration of an active ingredient. Examples, without limitation, of excipients include non-organic salts (e.g. calcium carbonate, calcium phosphate, potassium phosphate, sodium phosphate), bile salts (e.g. sodium deoxycholate, sodium taurocholate, glycodeoxycholat), phospholipids (e.g. lecithin, phosphatidylcholine, dipalmitoyl phophatidyl choline), biopolymers (e.g. chitosan and their derivatives), non-ionic surfactants (e.g. Poloxamer 188, cremophor EL, laurate sucrose ester (SE), and sucrose cocoate), alkylglycosides (e.g. tetradecylmaltoside (TDM) and N-lauryl-b-d-maltopyranoside), enzyme inhibitors (e.g. protease inhibitor), various sugars and types of starch, cellulose derivatives, gelatin, vegetable or synthetic oils, and polyethylene glycols.

Techniques for formulation and administration of drugs may be found in the latest edition of “Remington's Pharmaceutical Sciences,” Mack Publishing Co., Easton, PA, which is herein fully incorporated by reference.

Suitable routes of administration may, for example, include mucosal which includes, for example, nasal/intranasal, aerosol, buccal, sublingual and ocular; oral, which includes, for example, liquid; subcutaneous delivery; topical, which includes for example, gels and transdermal patches; intramuscular, suppository and intravenous delivery.

Pharmaceutical compositions of the present invention may be manufactured by processes well known in the art, e.g., by means of conventional mixing, dissolving, granulating, dragee-making, levigating, emulsifying, encapsulating, entrapping, thermal drying or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For transmucosal delivery (e.g., intranasal administration) the active ingredients for use according to the present invention can be delivered from a thin film/wafer or a gel. Aerosol spray from a pressurized pack or a nebulizer can be used for intranasal delivery. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch. The conjugate formulation for mucosal delivery may further contain one or more gelling agents, such that the oxytocin composition forms a gel in the nasal cavity, thus enhancing nasal absorption of oxytocin. Gelling systems useful in the compositions and methods described herein may include any known gelling system, such as a chemically reactive pectin-based gelling system (e.g., PecSys™, Archimedes Pharma) and a thermoreactive polymer gelling system (e.g., Pluronic®, F127, BASF). PecSys.™ is a low viscosity aqueous pectin-based solution, delivered as a fine mist in which each droplet gels on contact with calcium ions in the nasal mucosa. Other low methoxy pectin could also be employed. The gelling temperatures vary depending on the ratios of components and the amount of co-polymer employed in the final formulation. Gelling in the adult human nasal cavity has been demonstrated for Pluronic®. F127 at approximately 18-20% wt/vol, for examples, as used in a vitamin B12 gel supplement (EnerB, Nature's Bounty, NY) and in a gelling sumatriptan, which contains 18% wt/vol Pluronic® F127 and 0.3% wt/vol Carbopol (anionic bioadhesive polymer C934P). The monomer ratios and concentrations may be adjusted for the intended oxytocin formulations to ensure gelling at 25-37° C., around the typical temperature of 34° in the nasal cavity. If the gelation temperature is lower than 25° C., the formulation could gel at room temperature; if the gelation temperature is above 37° C. the formulation would not fully gel on contact with the nasal mucosa. In some embodiments, the oxytocin formulation or composition may further utilize a mucoadhesive agent such as Carbopol. Addition of a mucoadhesive, e.g., addition of up to 0.5% Carbopol, may further lower the gelation temperature.

The conjugate can alternatively be bound to a particulate carrier (microparticles, nanoparticles) such as oil drops, liposomes. The conjugate can be encapsulated/trapped or bound to a polymer such as a cyclodextrin via formation of inclusion complexes.

The bound conjugate can be surface-bound to the nanoparticles or sequestered within clefts/pockets formed in the nanoparticles. Use of a particulate carrier improves the bioavailability and transport of oxytocin to the therapeutic target.

According to one embodiment of the present invention a weight ratio of oxytocin, an oxytocin analog or derivative or an oxytocin receptor agonist to the fatty acid is 1:3000 to 1:200. In formulation containing a cyclodextrin such as 2-Hydroxypropyl-β-cyclodextrin, a weight ratio of the conjugate to the cyclodextrin can be 1:60,000 to 1:600.

Pharmaceutical compositions suitable for use in the context of the present invention include compositions wherein the active ingredients are contained in an amount effective to achieve the intended purpose. More specifically, a “therapeutically effective amount” means an amount of active ingredients effective to prevent, alleviate, or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient brain levels of the active ingredient to suppress symptoms associated with a neonatal disorder (i.e., minimally effective concentration, MEC). The MEC will vary for each preparation, but can be estimated from in vitro data or animal studies. Dosages necessary to achieve the MEC will depend on individual characteristics and route of administration. Detection assays can be used to determine concentrations.

Depending on the severity and responsiveness of the neonatal disorder to be treated, dosing can be of a single or a plurality of administrations, with course of treatment lasting from several days to several weeks, or until diminution of symptoms is achieved.

The amount of a composition to be administered will, of course, be dependent on the subject being treated, the severity of the affliction, the manner of administration, the judgment of the prescribing physician, etc.

Compositions of the present invention may, if desired, be presented in a dispenser device, such as an FDA-approved nebulizer, which may contain one or more unit dosage forms containing the active ingredient. The dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

Treatment of a neonate using the present conjugate can be effected as follows. Treatment frequency will be several times per day in one of the routes of administration (including but not limited to nasally, nebulizer, orally, intravenous, subcutaneous). Dose will be calculated per infant's weight, severity of NAS symptoms or the dose of narcotics for the treatment of NAS (including but not limited to methadone, morphine, buprenorphine).

Symptoms of NAS include: trembling, excessive crying or high-pitched crying, sleep problems, tight muscle tone, overactive reflexes, seizures, yawning, stuffy nose, and sneezing, poor feeding and sucking, vomiting or diarrhea, sweating, fever or unstable temperature. The Finnegan Neonatal Abstinence Scoring System is the most commonly used scoring tool.

Treatment with the present conjugate may have several effects:
(i) reduction of the frequency and severity of NAS-related symptoms (as described above) and overall Finnegan score;
(ii) reduction of the needed narcotics to treat NAS symptoms (including but not limited to methadone, morphine, buprenorphine);
(iii) earlier discharge from the hospital as a result of improvement of symptoms; and/or
(iv) reducing time to wean off narcotic treatment, time to wean off pain and other treatments of NAS symptoms, reduction of side effects and time to discharge.

As used herein the term “about” refers to ±10%.

Additional objects, advantages, and novel features of the present invention will become apparent to one ordinarily skilled in the art upon examination of the following examples, which are not intended to be limiting.

### EXAMPLES

Reference is now made to the following examples, which together with the above descriptions, illustrate the invention in a non limiting fashion.

### Example 1

### Pre-Formulation Development of Oxytocin Compositions for Intranasal Administration

The transport of peptides, such as human oxytocin, to the brain is significantly hindered by the blood-brain barrier (BBB). Intranasal administration appears to be an ideal solution for drug delivery to the brain, bypassing the BBB.

A unique drug delivery platform was developed based on loading of oxytocin onto/into lipid nanodroplets by forming an amide bond with a carboxyl group of fatty acid, e.g. DHA that is incorporated into a lipid core. When covalently bound to a fatty acid, oxytocin becomes more lipophilic, hence more adhesive to intranasal surfaces. In addition, the nanodroplets can be surrounded by non-ionic surfactants and cyclodextrins, which protect the drug molecule from enzymatic degradation and increase permeability.

Pre-formulation development was undertaken in order to address a predefined initial quality target product profile (QTPP), taking into account safety for the target group, properties of the drug substance and excipients, route of administration, dosage form, dosage strength, critical quality attributes, bioavailability and clinical efficacy.

The initial QTPP are provided in Table 1 below.

TABLE 1
Quality Target Product Profile (QTPP)

| Quality Attribute | Design Target |
|---|---|
| Therapeutic Indication | Neonatal Abstinence Syndrome (NAS) |
| Target population | Neonates |
| Route of administration | Non-invasive, nasal delivery |
| Dosage form | Solution or powder for reconstitution |
| Drug substance | Human Oxytocin |
| Dose | To be established upon pre-clinical development |
| Excipients | Compendial |
| | Safe to the target population |
| | Preservative free |
| Method of administration | Nasal administration, e.g. via spraying or inhalation. |
| Product quality attributes that | Identification |
| may affect clinical performance | Assay |
| | Degradation products |
| | Dose Uniformity |
| | pH |
| | Osmolality |
| | Microbial Limits |
| | Other attributes, relevant for the dosage form |
| | and formulation type (e.g. viscosity) |
| Container closure system | Suitable container closure system to ensure product |
| | safety, target shelf life and clinical usability. |
| Stability | Shelf life - at least 24 months |
| Storage conditions | For all climate zones: |
| | To be established according to the stability data. |
| | Initial stability programs: 5 ± 2° C., 25 ± 2° C. |

### Selection of Excipients

Excipients in a pediatric formulation should be chosen appropriately, avoiding any excipients that are potentially toxic or unsuitable for children. Choosing the right excipients in the development of a new pediatric drug is one of the most important aspects, as it requires special safety considerations [2,3]. The main principle in the choice of excipients was safety for the target group (neonates). For the preliminary development of the drug, only excipients with a proven safety profile were selected. All excipients are well known with respect to the pharmaceutical manufacturing, they are widely used in approved medicinal products for various routes of administration (ophthalmic, intravenous, oral) and their content in the proposed compositions is below the limits published in the FDA-IID (Inactive ingredient database).

### Nanodroplets Core

Fatty acids, saturated and polyunsaturated (PUFA), are known as absorption enhancers via the nasal and pulmonary route [4]. In the present formulation they are used as the base for the lipid core of the nanodroplets and as the loading site for oxytocin.

Unsaturated Omega-3 fatty acid-Cis-4,7,10,13,16,19-Docosahexaenoic acid (DHA FA) was chosen for pre-formulation studies due to its endogenous nature, proven safety profile and anti-inflammatory effect. In addition, DHA is essential for brain development of premature infants, so its penetration into the brain will provide additional therapeutic benefits.

### Surfactants

Non-ionic surfactants are the preferred choice for paediatric formulations. Non-ionic surfactants, consisting of a hydrophilic head group and a hydrophobic tail, carry no charge and are relatively non-toxic [4]. Tyloxapol and Lipoid E80 (Egg Yolk Lecithin) were used in preliminary studies, both surfactants are compendial with a proven safety profile.

### Stabilizer/Absorption Enhancer

Cyclodextrins can increase drug permeability by direct action on mucosal membranes and enhance intranasal drug absorption and/or bioavailability [4,5]. HPBCD was selected for the preliminary formulations because of its safety profile and ability to stabilize and protect the components of nanoemulsions in liquid or dry form (cryoprotection in freeze drying).

### Stability of Oxytocin

According to a literature review, the pH of commercially available oxytocin preparations ranges from 3.0 to 5.0, and the recommended storage temperature for finished formulations is 2-8° C. [6.7]. These recommendations will be taken into account when developing a nasal preparation.

### Loading of Oxytocin to Nanoparticles

Two oil in water nanoemulsions with oxytocin conjugated to fatty acid droplets via an amide bond were prepared using different production techniques (Examples 2 and 3).

The conjugation was performed via one-step coupling using crosslinking reagent N-(3-Dimethylaminopropyl)-N′-ethylcarbodiimide hydrochloride (EDC).

EDC reacts with carboxylic acid group oh DHA FA to form an active O-acylisourea intermediate (DHA-EDC) that is easily displaced by nucleophilic attack from primary amino groups of oxytocin in the reaction mixture. The primary amine forms an amide bond with the original carboxyl group, and an EDC by-product is released as a soluble urea derivative. The O-acylisourea intermediate is unstable in aqueous solutions. Failure to react with an amine results in hydrolysis of the intermediate, regeneration of the carboxyls, and the release of an N-unsubstituted urea. Reaction conditions, e.g. pH, temperature, reagents and products are described in EXAMPLES 1 and 2.

Released N-unsubstituted urea is removed from the formulation by tangential flow filtration (TFF).

### Example 2

### Conjugate Formulation

Emulsification of DHA free acid was performed using a low energy solvent displacement technique. Briefly, unsaturated fatty acid (DHA) and surfactant (Lipoid E80) were dissolved in ethanol and this organic solution was injected into constantly stirred water to form oil droplets (100-200 nm). The solvent was then removed under reduced pressure using a rotary evaporator and an emulsion was used for coupling of oxytocin using EDC*HCl. When the conjugated form of oxytocin (OXY-DHA) was formed and the O-acyl isourea intermediate (DHA*EDC) was completely deactivated, the reaction mixture was purified from unreacted peptide, coupling reagent and isourea by-product using tangential flow filtration (TFF) membrane, PES, MWCO 30,000.

The purified emulsion was mixed with HPBCD (10% w/v) and the pH was adjusted to 4.5.

### Emulsification

323 mg DHA free acid and 123 mg Lipoid E80 were weighed into a glass beaker and dissolved in 20 ml ethanol. The resulting solution was injected via 21G needle into 96 ml of continuously stirred (360 rpm) double deionized water (DDW), and then the solvent was removed under reduced pressure using a rotary evaporator (30 mbar). After evaporation, the weight of the emulsion was restored to 96 g with DDW; the average particle size was 154.3 nm, pH was 7.86.

### Loading of Oxytocin to Nanoparticles

50 ml of the resulting emulsion, containing 0.5 mmol DHA free acid, were used for the coupling reaction. The pH of the emulsion was adjusted to 4.5 with 1N hydrochloric acid and 0.1 mmol of EDC*HCl were added for activation of DHA carboxyl groups. The reaction mixture was stirred for 1 hour until complete activation.

21 mg (0.02 mmol) of human oxytocin was dissolved in 5 ml of DDW and the resulting solution was added to the reaction mixture. Initially, the pH was brought up to 8.5 and maintained in the range of 8.5-8.6 during reaction. The entire amount of oxytocin reacted within 1 hour (100% reaction yield). The reaction mixture was left overnight at 2-8° C. to deactivate DHA.

### Purification of Nanoemulsion

The deactivated emulsion was diluted with 4 volumes of DDW and filtered through a TFF membrane (PES, MWCO 30,000). The filtrate, containing unreacted coupling reagent and isourea by-product, was discarded. The retentate (38 ml) was taken for further processing.

### Formulating

18 g of HPBCD was dissolved in the retentate, the volume was adjusted to 180 ml with DDW and the pH was adjusted to 4.8. A portion of the final bulk was filled into glass vials (0.5 ml/vial) and lyophilized.

The finished product at both presentations, liquid and dry, was placed for stability testing at 2-8° C. and room temperature.

Test results for the liquid finished product: Oxytocin content 74 IU/ml, pH 5.5, osmolality 83 mosm/kg, average particle size 160.5 nm, Polydispersity index (PDI) 0.149.

Test results for the dry finished product: Oxytocin content 37 IU/vial, pH is 5.5, osmolality 76 mosm/kg, average particle size 147.2 nm, PDI 0.349.

The process yield in terms of oxytocin content was 100%.

TABLE 2
Composition of Formulation 1

| | | Content per |
|---|---|---|
| Component | Units | 1 ml |
| Oxytocin* | IU | 74.0 |
| DHA FA* | mg | 0.5 |
| Lipoid E80 | mg | 0.4 |
| HPBCD | mg | 100.0 |
| Water | ml | q.s. to 1 ml |

*Determined by HPLC

### Example 3

Conjugate Formulation Formulation 2 was prepared as follows: DHA free acid and Tyloxapol (water-soluble surfactant) were dispersed in DDW, and oxytocin was bound to the carboxyl groups using EDC*HCl.

Being conjugated to a fatty acid, Oxytocin begins to act as a hydrophobic compound that can be incorporated into lipid nanoparticles. Unreacted oxytocin, coupling reagent and its by-product are hydrophilic compounds with an affinity to aqueous media. Thus, the next production step is based on the polarity differences between the mixture components. The reaction mixture was mixed with DHA ethyl ester and Lipoid E80 and emulsified using a Microfluidizer LM20 through a 200 nm ceramic membrane at 15,000 psi.

The resulting emulsion contained nanodroplets with encapsulated Oxytocin; unwanted hydrophilic compounds remained in an aqueous medium. The nanodroplets were purified and concentrated using TFF and further formulated with HPBCD.

### Coupling of Oxytocin to DHA

326 mg DHA free acid and 120 mg Tyloxapol were dispersed in 100 ml DDW. A portion of 50 ml was taken for further processing. The pH of the mixture was adjusted to 4.5 with 1N hydrochloric acid; then 0.1 mmol of EDC*HCl was added for activation of DHA carboxyl groups. The reaction mixture was stirred for 1 hour until complete activation.

19.3 mg (0.02 mmol) of human oxytocin was dissolved in 2 ml of DDW and the resulting solution was added to the reaction mixture. Initially, the PH was brought up to 8.5 and maintained in the range of 8.5-8.6 during reaction. The entire amount of oxytocin reacted within 1 hour (100% reaction yield). The reaction mixture was left overnight at 2-8° C. to deactivate DHA.

### Emulsification

The deactivated emulsion was mixed with 120 mg of Lipoid E80 and 350 mg of DHA ethyl ester and emulsified using a Microfluidizer LM20 through a 200 nm ceramic membrane at 15,000 psi.

### Purification

The emulsion was diluted with 4 volumes of DDW and filtered through a TFF membrane (PES, MWCO 30,000). The filtrate, containing unreacted coupling reagent and isourea by-product, was discarded. The retentate (41 ml) was taken for further processing.

### Formulating

10 g of HPBCD was dissolved in 17 ml of retentate, the volume was completed to 100 ml with DDW and the pH was adjusted to 4.8.

A portion of the final bulk was filled into glass vials (0.5 ml/vial) and lyophilized.
The finished product at both presentations, liquid and dry, were placed for stability testing at 2-8° C. and room temperature.

Test results for the liquid finished product: Oxytocin content 55 IU/ml, pH 5.5, osmolality 84 mosm/kg, average particle size 168.9 nm, Polydispersity index (PDI) 0.239.

Test results for the dry finished product: Oxytocin content 27 IU/vial, pH is 5.5, osmolality 79 mosm/kg, average particle size 229.4 nm, PDI 0.292.

The process yield in terms of the oxytocin content is 100%.

TABLE 3
Composition of Formulation 2

| | | Content per |
|---|---|---|
| Component | Units | 1 ml |
| Oxytocin* | IU | 55 |
| DHA FA* | mg | 0.4 |
| DHA EE* | mg | 0.7 |
| Tyloxapol | mg | 0.2 |
| Lipoid E80 | mg | 0.4 |
| HPBCD | mg | 100.0 |
| Water | ml | q.s. to 1 ml |

*Determined by HPLC

### Feasibility Study for Nebulizing Oxytocin Nanoemulsions

A nebulizer is a device that converts liquid medicine into a fine aerosol. A feasibility study was carried out for the dispersion of oxytocin nanoemulsions through a nebulizer, suitable and approved for neonates and infants' use (Model: PARI BOY Junior)

1 ml of formulations 1 and 2 was loaded into a nebulizer and sprayed via silicon mask. The mask was then attached to a glass plate and the condensate was collected. The content of oxytocin in the initial formulation and condensate was determined using HPLC (Table 4).

TABLE 4

| | Content oxytocin | Content oxytocin | |
|---|---|---|---|
| | before nebulizing, | after nebulizing, | Recovery of |
| Sample | IU/ml | IU/ml | oxytocin, % |
| Formulation #1 | 77.1 | 73.6 | 96 |
| (Liquid) | | | |
| Formulation #2 | 58.0 | 58.1 | 100 |
| (Liquid) | | | |

The results of the feasibility study show that oxytocin nanoemulsions are suitable for nebulization and can be administered as aerosols.

### Example 4

### Testing of Oxytocin, Free Form

Approaches for testing the purity of the present formulations were developed. HPLC chromatography using a Thermo, Hypersil Gold-C18 column (3×50 mm, 3 μm), Cat. no. 25003-053030 with a gradient elution with water and acetonitrile (both acidified with 0.05% formic acid) was used to assess the purity of formulations 1 and 2.

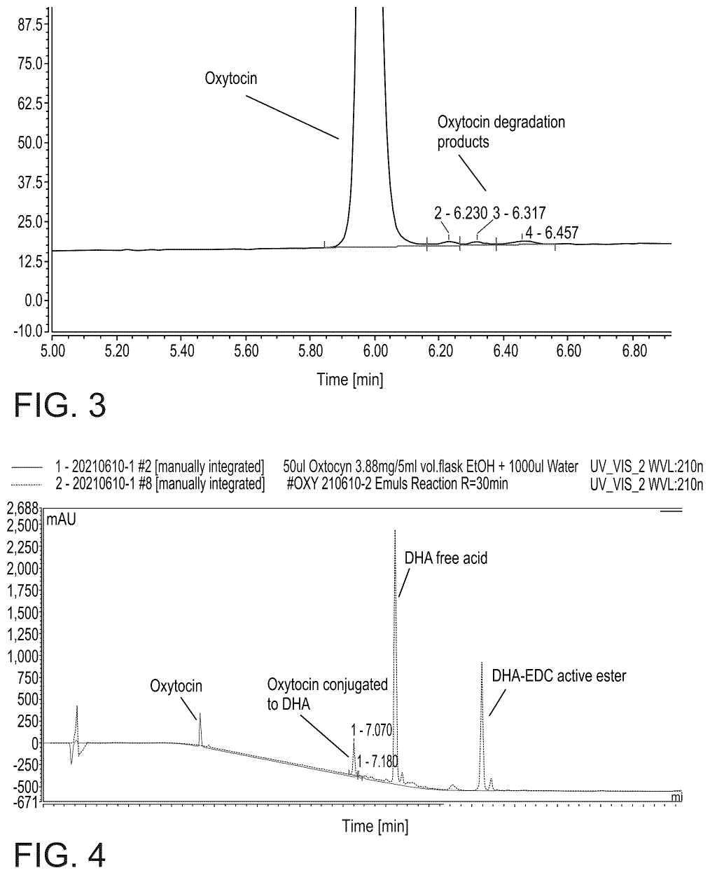

Reference solution of oxytocin and formulation samples were prepared by dissolving the substances in a 10% ethanol solution. The chromatograms are shown in FIGS. 2 and 3. HPLC conditions are outlines in Table 5 below.

TABLE 5

| Method parameter | Setting value | | |
|---|---|---|---|
| Column | Thermo, Hypersil Gold-C18 column | | |
| | (3 × 50 mm, 3 μm), | | |
| | Cat. no. 25003-053030 or equivalent | | |
| Oven | 45° C. | | |
| Autosampler temperature | 15° C. | | |
| Injection volume | 10 μl | | |
| Flow rate | 0.4 ml/min | | |
| UV detector | 210 nm | | |
| DAD | 200-280 nm | | |
| Mobile Phase A | 1000 ml Water:0.5 ml Formic acid | | |
| Mobile Phase B | 1000 ml Acetonitrile:0.5 ml Formic acid | | |
| | Time | % A | % B |
| Gradient | 0 | 94 | 6 |
| | 5 min | 72 | 28 |
| | 7 min | 55 | 45 |
| | 9 min | 55 | 45 |
| | 9.1 min | 94 | 6 |
| | 14 min | 94 | 6 |

| | |
|---|---|
| Run time | 14 min |
| Retention time | Oxytocin = 5.9 min |

### Example 5

### Testing of Oxytocin Conjugate

The formulations described in Example 2 and 3 were tested using the HPLC procedure outlined in Table 6 below.

TABLE 6

| Method parameter | Setting value | | |
|---|---|---|---|
| Column | Thermo, Hypersil Gold-C18 column | | |
| | (3 × 50 mm, 3 μm), | | |
| | Cat. no. 25003-053030 or equivalent | | |
| Oven | 45° C. | | |
| Autosampler temperature | 15° C. | | |
| Injection volume | 10 μl | | |
| Flow rate | 0.5 ml/min | | |
| UV detector | Oxytocin - 210 nm | | |
| | DHA - 220 nm | | |
| DAD | 200-280 nm | | |
| Mobile Phase A | 1000 ml Water:0.5 ml Formic acid | | |
| Mobile Phase B | 1000 ml Acetonitrile:0.5 ml Formic acid | | |
| | Time | % A | % B |
| Gradient | 0 | 94 | 6 |
| | 1.0 min | 86 | 14 |
| | 7.0 min | 2 | 98 |
| | 12.5 min | 2 | 98 |
| | 12.6 min | 94 | 6 |
| | 17.0 min | 94 | 6 |

| | |
|---|---|
| Run time | 17.0 min |
| Retention time | Oxytocin = 3.7 min |
| | OXY-DHA = 7.0 min |
| | DHAFA = 8.0 min |
| | DHA-EDC = 10.0 min |

### Monitoring of Coupling Reaction

The reaction mixture consisting of Oxytocin, Oxytocin conjugated to DHA, DHA, activated DHA (DHA-EDC) was monitored by the HPLC detector at UV spectrum wavelengths.

All the components of the reaction mixture were well separated as is shown by the chromatograms of FIG. 4.

### Testing of Finished Product

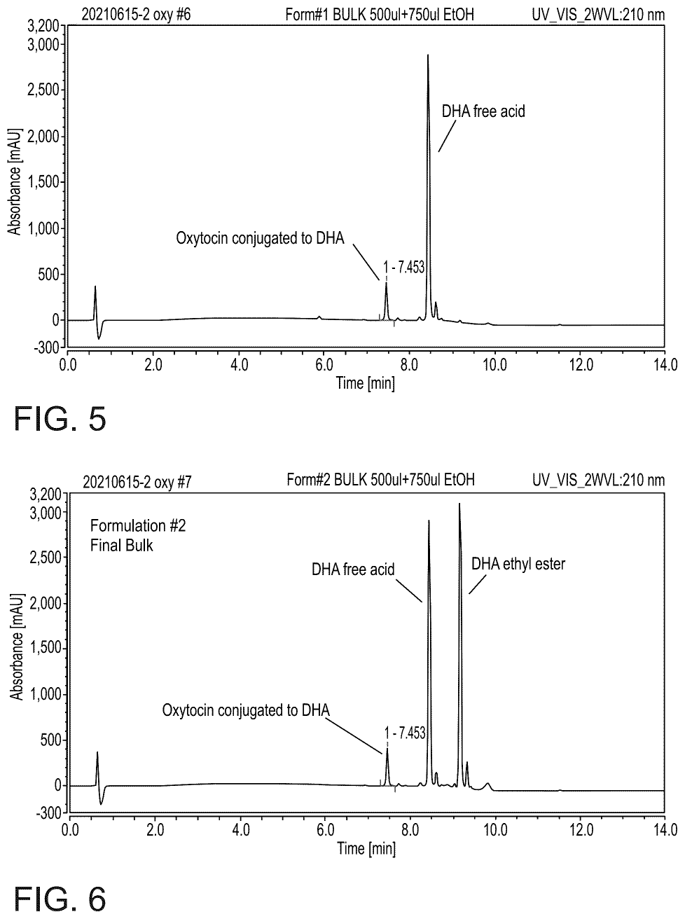

The formulations described in Examples 1 and 2 were diluted with ethanol 1:1.5 and tested by HPLC. Typical chromatograms of Formulations 1 and 2 are shown in FIGS. 5 and 6 respectively.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

In addition, any priority document(s) of this application is/are hereby incorporated herein by reference in its/their entirety.

### REFERENCES

1. Bonferoni M. C., Silvia Rossi S. et al., Nanoemulsions for “Nose-to-Brain” Drug Delivery. J. Pharmaceutics, v. 11 (2); 2019.
2. Rouaz K. et al., Excipients in the Paediatric Population: A Review. J. Pharmaceutics 2021, 13, 387.
3. European Medicines Agency (EMA); Committee for Medicinal Products for Human Use (CHMP); Paediatric Committee (PDCO). Guideline on Pharmaceutical Development of Medicines for Paediatric Use. Available online: www(dot)ema(dot)europa(dot)eu/en/documents/scientific-guideline/guideline-pharmaceutical-development-medicines-paediatric-use_en(dot)pdf (accessed on 1 Jun. 2020).
4. Ghadiri M., Young P. M., Train D. Strategies to Enhance Drug Absorption via Nasal and Pulmonary Routes. J. Pharmaceutics 2019, 11, 113.
5. European Medicines Agency (EMA); Committee for Medicinal Products for Human Use (CHMP); EMA/CHMP/333892/2013. Background review for cyclodextrins used as excipients.
6. Revisiting the Stability and Storage Specifications of Oxytocin Injection: A Literature Review. PQM; July 2018.
7. Hawe A. et al., Towards Heat-stable Oxytocin Formulations: Analysis of Degradation Kinetics and Identification of Degradation Products. Pharmaceutical Research, Vol. 26, No. 7, July 2009.
8. Davis, J. M., Shenberger, J., Terrin, N., Breeze, J. L., Hudak, M., Wachman, E. M., . . . & Lester, B. (2018). Comparison of safety and efficacy of methadone vs morphine for treatment of neonatal abstinence syndrome: a randomized clinical trial. JAMA pediatrics, 172 (8), 741-748.

1. A composition-of-matter comprising a conjugate of oxytocin, an oxytocin analog or derivative or an oxytocin receptor agonist bound to a fatty acid.

2. The composition-of-matter of claim 1, wherein said fatty acid is Docosahexaenoic acid (DHA).

3. The composition-of-matter of claim 2, wherein said DHA is bound to said oxytocin, oxytocin analog or derivative or an oxytocin receptor agonist through an amide linkage.

4. The composition-of-matter of claim 2, wherein a weight ratio of oxytocin, an oxytocin analog or derivative or an oxytocin receptor agonist to fatty acid is 1:3000 to 1:200.

5. The composition-of-matter of claim 1, further comprising a cyclodextrin.

6. The composition-of-matter of claim 5, wherein said conjugate is trapped in or bound to, an inclusion complex formed from said cyclodextrin.

7. The composition-of-matter of claim 6, wherein said cyclodextrin is (2-Hydroxypropyl)-β-cyclodextrin.

8. The composition-of-matter of claim 4, wherein a weight ratio of said conjugate to said cyclodextrin is 1:60,000 to 1:600.

9. The composition-of-matter of claim 1, further comprising liposomes encapsulating said conjugate.

10. A pharmaceutical composition comprising a conjugate including oxytocin, an oxytocin analog or derivative or an oxytocin receptor agonist bound to a fatty acid and a carrier for mucosal delivery.

11. The pharmaceutical composition of claim 10, wherein said carrier is formulated for nasal delivery.

12. The pharmaceutical composition of claim 10, further comprising a cyclodextrin forming an inclusion complex.

13. The pharmaceutical composition of claim 12, wherein said conjugate is trapped in or bound to said inclusion complex.

14. The pharmaceutical composition of claim 10, further comprising liposomes encapsulating said conjugate.

15. A nebulizer for nasal delivery comprising the pharmaceutical composition of claim 10.

16-18. (canceled)

19. A method of reducing the need and dosage of narcotics prescribed to an infant, by delivering a conjugate including oxytocin, an oxytocin analog or derivative or an oxytocin receptor agonist bound to a fatty acid to a neonate.

20-21. (canceled)

22. The method of claim 19, wherein said conjugate is trapped in or bound to an inclusion complex formed by a cyclodextrin.

23. The method of claim 19, wherein said conjugate is encapsulated by a liposome.

24. The method of claim 19, wherein delivery is effected using a nebulizer.
